SEQUENCE LISTING (2 sequences)

SEQ ID NO: 1 - protein, 17 aa, Artificial Sequence
  Description of Artificial Sequence synthetic haptoglobin peptide
VETGSEATDIEDDSSAK

SEQ ID NO: 2 - protein, 21 aa, Artificial Sequence
  Description of Artificial Sequence synthetic haptoglobin peptide
SRQFYRLRTEGDGVYTLNSEK

(19) United States
(12) Patent Application Publication
(10) Pub. No.: US 2003/0158391 A1
(43) Pub. Date: Aug. 21, 2003

(54) Anitbodies and method for producing same, the use thereof and immunisation cocktails, immunoassays-sets and peptides

(76) Inventor: Mark Hennies, Oberursel (DE)

Correspondence Address: BIRCH STEWART KOLASCH & BIRCH, PO BOX 747, FALLS CHURCH, VA, 22040-0747, US

(21) Appl. No.: 10/148,636

(22) PCT Filed: Dec. 15, 2000
(86) PCT No.: PCT/EP00/12798

(30) Foreign Application Priority Data
Dec. 15, 1999 (DE) 199 60 500.9

(51) Int. Cl. C07K 16/20; A61K 39/395; C12P 21/08; C12P 21/04

(52) U.S. Cl. 530/388.1; 424/130.1; 435/70.21

(57) ABSTRACT

The present invention relates to a process for preparing protein-specific antibodies, to protein-specific antibodies which can be obtained thereby, namely polyclonal, crossreactive antisera possessing specificity for haptoglobin derived from at least two productive animal and domestic animal species, and to their use for determining haptoglobin in productive animal and domestic animal samples. The invention furthermore relates to corresponding immunization cocktails, to peptides which can be used for preparing the antisera, and to immunoassay sets which are based on the antisera. Both protein and protein fragments are used for the immunization.

The present invention relates to a process for preparing protein-specific antibodies, to protein-specific antibodies which can be obtained thereby, namely polyclonal, crossreactive antisera possessing specificity for haptoglobin from at least two productive animal and domestic animal species, and to their use for determining haptoglobin in productive animal and domestic animal samples. The invention furthermore relates to immunization cocktails containing protein and at least one protein fragment, to peptides which can be used for preparing the antisera, and to immunoassay sets which are based on the antisera.

Important diagnostic information with regard to inflammatory, infectious or traumatic disease states of a clinical and also subclinical nature is increasingly based on determining acute phase proteins. The proteins are plasma proteins whose concentration varies with the response of a host to an infection, an inflammation or a traumatic event. In this connection, the increase in concentration in the plasma can be more or less pronounced, from a very large 100-fold to 500-fold increase, by way of a 3-fold to 4-fold increase of a moderate response, and through to a plasma level, which has only increased 2-fold, of a relatively weak response, cf. Eckersall et al., Veterinary Medicine, Vol. 41, 643 (1999).

Haptoglobin belongs to the group of acute phase proteins. In cattle, the haptoglobin concentration increases during the acute phase response, starting from a concentration of less than 0.01 g/l and increasing to a concentration of 2-3 g/l within 48 h after infection, i.e. representing an increase of up to 300-fold. In pigs, on the other hand, only a moderate increase, of from about 1-2 mg/ml to about 5 mg/ml, is seen following the injection of turpentine (Eckersall et al., 1999).

Methods for quantitatively determining haptoglobin are based either on biochemical methodology or immunological methodology. Since the biochemical measurement method makes use of the binding of haptoglobin to hemoglobin, even the slightest traces of hemoglobin can adversely affect the accuracy of the measurement.

Immunological methods of measurement require suitable antibodies to be prepared first of all. Thus, in Veterinary Immunology and Immunopathology, 51, 277-292 (1996), Godson et al. describe an ELISA in which a monoclonal antibody directed against bovine haptoglobin is used as the capture antibody and a rabbit antiserum directed against bovine haptoglobin is used as the detection antibody. Haptoglobin concentrations in horse sera have been determined immunoturbidimetrically using a sheep antiserum directed against horse haptoglobin (Kent and Goodall, Equine Veterinary Journal, 23 (1), 59-66 (1991)). In Comp. Biochem. Physiol., Vol. 119B, 2, 365-373 (1998), use is made of polyclonal antibodies which are directed against human haptoglobin, and which are prepared in rabbits and goats, to determine haptoglobin in pigs. In Research in Veterinary Science, 63, 145-149 (1997), McNair et al. describe a competitive immunoassay which makes use of a europium-conjugated monoclonal antibody which has specificity for bovine haptoglobin. An antibody of this nature is also used in the investigation, carried out by Young et al. in Veterinary Immunology and Immunopathology, 49, 1-13 (1995), with regard to validating immunoassays for bovine haptoglobin.

Despite any cross reactivity which the antibodies employed may possess, the immunoassays which have thus far been described are orientated toward animal species specificity since it is generally assumed that only species-specific antisera and standards are able to meet the demands for consistent and comparable immunochemical tests (Eckersall et al., 1999).

However, the sensible use of a quantitative haptoglobin determination in veterinary medical diagnosis requires the establishment of a test method which can be used to investigate various animal species equally, with approximately the same sensitivity, by taking into consideration any possible animal species-specific variation ranges.

The preparation of a polyclonal antiserum normally starts with an active immunization, that is the artificial generation of an immune response as a defense mechanism within a host organism. For this, the antigen in question is administered to the host, with this in turn stimulating antigen-reactive B cells to expand and differentiate. Normally, antibodies directed against a protein, as the antigen, are obtained by immunizing with the protein or with synthetic polypeptides.

Ideally, the pure antigen is used for preparing a specific antibody. If the high degree of purity which this requires cannot be achieved, or can only be achieved with difficulty, it is also possible, where appropriate, for synthetic peptides, possessing constituent sequences for the protein, to lead to antipeptide antibodies which crossreact with the complete protein.

The object underlying the invention, i.e. to make it possible to determine immunologically proteins, in particular haptoglobin, from different animal species, in particular from at least two productive animal and domestic animal species, is achieved, according to the invention, by means of a special process for preparing protein-specific, in particular haptoglobin-specific antibodies. This process uses at least one immunization cocktail which contains both protein and fragments of the protein.

The present invention therefore relates to a process for preparing protein-specific antibodies by immunizing a host and, in a manner known per se, obtaining the antibodies, characterized in that at least one immunization cocktail, which contains protein and at least one protein fragment, is administered to the host.

Within the meaning of the invention, protein-specific antibodies are antibodies which bind specifically to a particular protein. In this connection, the term protein encompasses polypeptides which may be essentially homogeneous structurally, but which may also be a mixture composed of structurally different polypeptides, for example different phenotypes and isotypes of a protein, different species-specific variants of a protein, which variants originate from evolutionary divergence, or individual protein variants which mainly originate from mutations. In this respect, protein-specific antibodies within the meaning of the invention may also be crossreactive antibodies which are polyclonal or monoclonal and which possess specificity for at least two structurally different proteins. Structural differences are to be understood as being in the sense of antigen recognition and include different sequences in exactly the same way as different spatial structures, such as conformities or derivatizations, e.g. glycosylations.

Immunization means the generation of an immune response, as a defense mechanism in an organism, against an antigen. One or more antigens is/are administered to the host, with the antigen(s) preferably being injected into the bloodstream or into a tissue. In this respect, the generation of an immune response is artificial; for this reason, it is referred to as being an active immunization. As a rule, the immunization is effected in accordance with a predetermined immunization scheme. The primary immunization is advantageously followed by further immunizations, for the purpose of generating immunological secondary reactions.

The number of the immunizations to be performed depends, first and foremost, on the host and on the development of the antibody titer in each particular case. According to the invention, preference is given to performing more than two and, in particular, three to five, immunizations. According to a particular embodiment of the present invention, four immunizations are performed.

As a rule, the individual immunizations are effected at intervals of several weeks, which intervals should advantageously not exceed two months in length. Preference is given to the immunization intervals being of from two to seven weeks in length, in particular of from three to six weeks in length. According to an advantageous embodiment of the present invention, the intervals between the individual immunizations are about five weeks in length.

If several immunizations are performed, the immunization cocktails which are administered at the time of the individual immunizations may be identical or, preferably, different. According to the invention, at least one immunization cocktail comprises protein and at least one protein fragment whereas either protein or at least one protein fragment can be administered, in a conventional manner, by means of the other immunization cocktails. However, it is advantageous if all the immunization cocktails which are used within a process comprise protein and at least one protein fragment, with it being possible, however, for these immunization cocktails to differ in their respective compositions, which is something which is preferred in particular cases.

As a rule, the host which is employed is a higher vertebrate. Preference is given to rodents, in particular rabbits, mice, rats and hamsters, and also goats and sheep. According to an advantageous embodiment of the present invention, rabbits are used as the host.

The antibodies which are formed can be isolated, in a manner known per se, for example from the spleen, from lymph nodes and from peripheral blood. In order to obtain polyclonal antisera, blood is preferably withdrawn from the host and the serum then isolated from this blood. This serum, which is termed complete serum, is only partially composed of immunoglobulins, which means that further purification steps for enriching the desired antibodies may follow. As is known, use can be made, for this purpose, of salting out, for example by means of fractional ammonium sulfate precipitation, ultrafiltration, rebuffering, dialysis, precipitation by dialysis against acid buffers, various chromatographic methods, such as ion exchange chromatography, gel permeation chromatography, hydrophobic chromatography and affinity chromatography, etc. Advantageously, high-titer complete sera are obtained over an appropriate period of time, as a rule several weeks or months, with the host finally being exsanguinated.

In order to obtain monoclonal antibodies, preference is given to the spleen tissue being removed from the host and the spleen lymphocytes, i.e. antibody-producing B cells, which are contained therein being fused with myeloma cells, then suitable hybridomas, which produce a desired antibody, being selected and cloned and subsequently employed for producing monoclonal antibodies. The respective steps of the procedure are known to a skilled person.

Furthermore, the antibodies which have been obtained in this way, in particular the monoclonal antibodies, can be modified; fragments of these antibodies, or chimeric antibodies, can be produced, depending on the nature of the task and the area of employment. Suitable techniques are known to the skilled person.

According to the invention, an immunization cocktail (vaccine) is understood as meaning a mixture composed of antigen and of auxiliary substances, in particular adjuvant, which are to be used if required. Adjuvants are substances into which the antigen is incorporated, or which are at least injected together with the antigen, and which augment the immune response. This includes depot-forming, macrophage-activating and/or lymphocyte-influencing adjuvants. As a rule, these adjuvants contain oils, such as paraffin oil, squalene or fatty acid esters such as mannide monooleate, sorbitan monooleate, etc., for the purpose of forming oil-in-water or water-in-oil emulsions of the antigen. Adjuvants can also contain killed pathogens or toxins or constituents thereof, such as killed Mycobacterium tubercolosis, pertusis vaccine, detoxified monophosphoryl lipid A from S. Minnesota, cell wall constituents from the tubercle bacillus, etc. According to the invention, preference is given to Freund's adjuvant, in particular complete Freund's adjuvant for the initial immunization and incomplete Freund's adjuvant for subsequent immunizations.

An antigen according to the invention is composed of protein and at least one protein fragment. Expediently, the protein possesses at least one antigenic determinant against which the protein-specific antibodies which are to be prepared are directed. As a rule, at least a part of the protein which is to be employed as the antigen corresponds to at least a part of the protein against which the protein-specific antibodies are directed. The protein which is to be employed in the process can be a polypeptide fraction which is essentially homogeneous structurally; however, preference is given to a mixture of structurally different polypeptides, in particular a mixture of species-specific variants of a protein. For further explanation of this feature, the reader may be referred correspondingly to the above remarks with regard to the term protein.

A protein which is suitable in accordance with the invention can be obtained from natural sources. Another possibility is that of preparing it recombinantly. In either case, the skilled person is familiar with the relevant techniques from protein biochemistry and molecular biology. Preference is given to a protein which is as pure as possible.

Protein fragments which can be used in accordance with the invention are preferably derived from the protein against which the protein-specific antibodies are directed. Use is advantageously made of at least one antigenic fragment. As a rule, the protein fragments are, where appropriate, derivatized peptides whose amino acid sequences at least include parts which are part of one or more polypeptides of the protein. Peptides having a length of from 10 to 30 amino acids, preferably of from 15 to 25 amino acids, are advantageous. For reasons of antigenicity, peptides which are particularly useful in accordance with the invention are derived from surface regions, which are preferably glycosylation-free, and possess a high antigenicity index. Particular preference is given to peptides whose sequence differs from homologous sequences which may possibly be present in the host. The immunization cocktail contains at least one fragment type, advantageously two or more different fragments.

Protein fragments which can be used in accordance with the invention can be prepared in a manner known per se. Peptides can, for example, be obtained from natural sources or synthesized from corresponding amino acids. A protein which can be used in accordance with the invention and which can, for example, be digested enzymically presents itself as a natural source. The resulting fragments can be worked up and interesting fragments can be isolated and, if necessary, modified. A peptide is preferably synthesized chemically, in particular using known solid-phase synthesis systems. As a rule, functionalized resin, to which the peptides being synthesized can be linked, are used for this purpose. Current examples are Wang resins for FMOC solid-phase peptide synthesis or Merrifield resins for BOC solid-phase peptide synthesis. After a desired peptide has been synthesized, it is then released from the support, with it being possible to eliminate any protecting groups which may be present either at the same time or subsequently. A subsequent purification, which may, where appropriate, be necessary, for example by means of HPLC, yields the peptide in the desired purity, as can be established, for example, by means of mass spectroscopy or NMR.

Immunization cocktails which can be used in accordance with the invention can contain protein and protein fragments as a physical mixture, i.e. protein and fragments, and also any adjuvants which may be present, are mixed with each other in any arbitrary sequence. However, preference is given to at least a part of the fragments being coupled to at least a part of the protein. According to the invention, coupling is understood as meaning any molecular interaction which leads to the fragments being bound to the protein such that a particular spatial arrangement with regard to each other is achieved. The interactions may be based on covalent, metal complex-like, coordinate or hydrophobic bonds, hydrogen bonds, electrostatic attractive forces, van der Vaals forces, or the like. According to the invention, preference is given to covalent bonds. The interactions between the proteins and the protein fragments can be direct or indirect. As a rule, indirect interactions are mediated by way of linkers which may be homobifunctional or hetereobifunctional. Use is advantageously made of the reagents which are customarily employed for conjugating or crosslinking proteins, for example maleimidocarboxylic acid NHS esters, such as maleimidoacetic acid NHS ester, m-maleimidobenzoic acid NHS ester, etc., 3-(2-pyridyldithio)propionic acid NHS ester, and the like. Preference is given to glutaraldehyde. The skilled person is very familiar with dealing with these reagents; for example, the protein and peptide can be reacted, at room temperature, with an aqueous solution of glutaraldehyde, with an efficient coupling already being achieved after a few hours.

Where the protein fragments are not themselves immunogenic, it is also advantageous to couple those fragments which are not coupled to protein which is employed, according to the invention, as antigen, to other carrier substances, in particular carrier proteins. This is primarily the case when peptides containing less than about 30 amino acids are to be used as protein fragments. Insofar as the carrier substances are also proteins, the above comments with regard to coupling protein and protein fragments apply correspondingly. If carrier substances of another constitution are used, the coupling then naturally depends on the functional groups which are present on the carrier substance. Homobifunctional and heterobifunctional linkers can also be obtained in these cases.

Preference is given to the carrier substances being strongly immunogenic carrier substances which are able to induce antibodies efficiently. These substances include, for example, various albumins, such as bovine serum albumin, ovalbumin or human serum albumin, globulins, such as thyroglobulin, etc. Preference is given to the hemocyanin of the horseshoe crab Limulus polyphemus, also termed keyhole limpet hemocyanin (KLH).

According to a particular embodiment of the present process, in the case of at least one immunization cocktail, all the protein fragments which are used for this immunization cocktail are included in the coupling reaction with the protein which is used as the antigen in the immunization cocktail. Provided the coupling reaction is complete, all the fragments are consequently coupled to at least a part of the protein. Alternatively, the part of the fragments which is not coupled to the protein can be coupled to another carrier substance such that all the protein fragments are coupled to a carrier substance, namely one part to the protein and another part to another carrier substance, which is preferably also a protein.

The advantageous possibility of being able to use different immunization cocktails within a process according to the invention, gives rise to the possibility of making variations, for example, in the case of the protein component, with regard to concentration, degree of coupling, nature of the protein, for example a protein from only one animal species or from several animal species, and different choice of the animal species, and, in the case of the protein fragments, with regard to the nature and, where appropriate, number of different fragments, their concentration and the nature of the coupling partner and also the ratio of fragment(s) to protein.

According to a particular embodiment of the present invention, immunization cocktails which are administered in an early phase of the immunization contain uncoupled protein, conjugates composed of protein and at least one protein fragment, and also conjugates of at least one protein fragment with other carrier proteins. While, within the context of this embodiment, immunization cocktails which are administered in a late phase of the immunization preferably contain uncoupled protein and conjugates of the protein with at least one protein fragment, they do not contain any conjugates of protein fragments with another carrier protein. The early phase of the immunization can mean, for example, the initial immunization and the second immunization, while the third immunization and the fourth immunization, for example, belong to the late phase of the immunization.

According to another particular embodiment of the present invention, the immunization cocktails which are administered in the early phase of the immunization contain higher quantities of protein fragments than do the immunization cocktails which are administered in a late phase of the immunization cocktail.

According to another particular embodiment of the present invention, the host is administered immunization cocktails which contain protein from at least two different animal species and whose protein components differ with regard to number and/or nature of the animal species-specific protein variants employed.

Within the context of the particular embodiments explained above, preference is given to using at least two different protein fragments, with the individual fragment types advantageously being treated separately from each other with regard to coupling/noncoupling.

A particular embodiment of the present invention relates to a process for preparing haptoglobin-specific antibodies by immunizing a host and obtaining the antibodies which have been formed, which process is characterized in that the host is administered at least one immunization cocktail which contains haptoglobin and at lest one haptoglobin fragment.

Haptoglobin (abbreviated to Hp) is understood as meaning a group of chemically related glycoproteins which are present in blood plasma and which possess a specific ability to bind to hemoglobin (Hb). In the sense of the previously defined term “protein”, according to the invention, the term “haptoglobin” consequently represents all the haptoglobin phenotypes, isotypes and variants. Haptoglobins from various animal species are included, as are mutated haptoglobins or haptoglobins which have been modified in some other way. The term includes, in particular, animal species-specific haptoglobins from humans and all productive animal and domestic animal species, that is from mammals, in particular from rodents, for example mice, rats, hamsters, hare-like mammals, for example rabbits, dogs, cats, artiodactyla, for example pigs and cattle, perissodactyla, for example horses, and primates.

Haptoglobin which can be used in accordance with the invention may be of natural or recombinant origin. Haptoglobins of natural origin can be obtained, for example, from blood components such as sera or plasma. For example, haptoglobin can be obtained from human plasma. A product of this nature can be obtained commercially as a lyophilized powder which is essentially salt-free. Haptoglobins can also be obtained in a similar form from other animal species; purification methods which are suitable for this purpose are known. It is advantageously possible to use nonhemolytic sera, resulting in particularly high yields being obtained. The following methodological procedures have proved to be particularly suitable:

Ammonium sulfate precipitation. The optimal concentration for selectively precipitating haptoglobin can be determined by means of a preliminary experiment.

Purification by affinity chromatography. For this, hemoglobin, preferably bovine hemoglobin, is coupled to a solid phase, for example to activated CNBr-Sepharose.

Gel filtration. Matrices composed of crosslinked dextran (Sephadex®), crosslinked polymer of allyldextran and N,N-methylenebis(acrylamide)(Spephacryl®), agarose (Sepharose®), highly crosslinked agarose (Superose®), and similar gel-forming materials, can be used for separating off impurities, as desired. Crosslinked matrices composed of agarose and dextran (Superdex®) are preferred.

According to the invention, the haptoglobin is then used for the immunization. In addition to this, it is used as a standard in immunoassays, preferably in the form of the haptoglobin which is specific for the species in which haptoglobin is to be determined.

According to a particular embodiment of the process for preparing haptoglobin-specific antibodies, the immunization cocktails contain mixtures of haptoglobins from different animal species, preferably from pig, bovine, horse, dog and/or human. Preference is given to administering, during the course of a process, immunization cocktails whose haptoglobin composition differs. Thus it is possible, for example, to administer immunization cocktails which, on the one hand, contain a selection of three or four of the abovementioned haptoglobins and, on the other hand, contain all the abovementioned haptoglobins. The former immunization cocktails are preferably administered at a comparatively early point in time, while the latter are preferably administered toward the end of the immunization. The selection can also be varied with regard to the number and/or type of the haptoglobins employed.

According to a particular embodiment of this process, the first immunization cocktail contains haptoglobins from pig, bovine, horse and human, while the second and the third immunization cocktails contain haptoglobins from pig, dog, horse and human and the fourth immunization cocktail contains haptoglobins from pig, bovine, dog, horse and human.

As a rule, the protein fragments employed are haptoglobin peptides. Peptides which are particularly suitable exhibit a relatively high degree of homology with the corresponding sequences of haptoglobins from different animal species and, in particular, those animal species whose haptoglobins are to be recognized specifically by the antibodies which are to be prepared. Preference is given to a degree of homology of at least 80%, in particular of at least 90%, and, particularly preferably, of virtually 100%. Peptide sequences which can be used in accordance with the invention comprise conserved regions of haptoglobins, i.e. their sequence is essentially animal species-nonspecific. Peptides which are not part of the hemoglobin-binding site are advantageous.

As a rule, suitable peptides have a length of from 10 to 30 amino acids, preferably of from 15 to 25 amino acids. Examples of such peptides, which have proved to be particularly suitable, are the amino acid sequences SEQ ID NO:1 and SEQ ID NO:2. These peptides are likewise part of the subject matter of the present invention.

If the protein fragments which are to be used are not immunogenic, or only weakly immunogenic, their immunogenicity can be increased by coupling them to carrier substances. A number of coupling possibilities, some of which have been explained above, are available to the skilled person for this purpose. According to the invention, preference is given to reacting with glutaraldehyde, for example by incubating the haptoglobin or haptoglobin mixture together with the peptide or peptide mixture in water or an aqueous solvent. This reaction can conveniently be carried out at ambient temperature, that is room temperature as a rule. However it can also be expedient to cool or to warm slightly. As a rule, the reaction leads to the desired result within a few hours; a reaction duration of 2 h, for example, is in the customary range. As a rule, the glutaraldehyde concentration is in the ppm to % range, expediently from 10 ppm up to 1%, preferably from 100 ppm up to 0.5%. It is within the ability of the skilled person to optimize the reaction parameters.

According to the invention, at least a part of the haptoglobin fragments is coupled to at least a part of the haptoglobin. If a haptoglobin mixture composed of haptoglobins from different animal species is used, the haptoglobin fragments can then be coupled to haptoglobin derived from all the animal species employed or else to a selection of haptoglobins derived from individual animal species. If a mixture of different haptoglobin fragments, for example a peptide mixture, is used, these fragments can then be coupled without differentiating in accordance with the fragment type, or else particular fragment types can be assigned to particular coupling partners.

According to the invention, useful conjugates composed of haptoglobin and haptoglobin fragments thereof can be obtained by reacting haptoglobin, preferably a mixture of haptoglobins derived from different animal species, with one or more haptoglobin fragments, preferably one or more haptoglobin peptides. If different fragments are used, this reaction is then carried out for each fragment type, preferably separately from each other.

As explained above, it is not absolutely necessary to couple the entire quantity of haptoglobin fragments to haptoglobin. If the fragments are not immunogenic, or only weakly immunogenic, it is then in this case advantageous to couple the fragments which are not coupled to haptoglobin to another carrier substance, preferably to a carrier protein. KLH is preferred in connection with haptoglobin. The reader is referred, mutatis mutandis, to the comments made above with regard to the coupling reaction.

According to a particular embodiment of this process, immunization cocktails which are administered in an early phase of the immunization contain uncoupled haptoglobin, at least one haptoglobin-peptide conjugate and at least one KLH-peptide conjugate. Immunization cocktails which are administered in a late phase of the immunization contain uncoupled haptoglobin and at least one haptoglobin-peptide conjugate, but no KLH-peptide conjugate. The haptoglobin-peptide conjugates are preferably mixtures of conjugates with haptoglobins derived from different animal species. Preference is likewise given to using different peptides, such that it is possible to refer to a conjugate mixture in this regard as well. If, for example, four immunizations are performed, immunization cocktails containing haptoglobin-peptide conjugate and KLH-peptide conjugate can be administered in association with the first and the second immunization, whereas the immunization cocktails which are administered in association with the third and the fourth immunizations then contain haptoglobin-peptide conjugate but no KLH-peptide conjugate. In addition, the immunization cocktails preferably contain uncoupled haptoglobin or a mixture of uncoupled haptoglobins derived from different animal species.

Consequently, the antigen used in the immunization cocktails is composed of several components, for example of uncoupled haptoglobin, or a mixture of uncoupled haptoglobins from different animal species, of one or more haptoglobin-peptide conjugates and/or one or more KLH-peptide conjugates.

In order to prepare the immunization cocktails, the components which are to be used are initially added to each other. It is advantageous to firstly incubate the resulting component mixture. This is conveniently effected at ambient temperature, that is at room temperature as a rule. However, it may be expedient to cool or slightly heat the mixture. As a rule, the incubation lasts from a few minutes up to a few hours; an incubation time of 1 h has proved to be advantageous.

In addition to the antigen, immunization cocktails as a rule contain other auxiliary substances, in particular adjuvants which are customarily employed for immunization. In the context of this embodiment, preference is given to using Freund's adjuvant. In particular, complete Freund's adjuvant is used for the first immunization whereas all the subsequent immunizations are carried out using incomplete Freund's adjuvant. In order to prepare the immunization cocktails, the antigen, preferably as the above-described component mixture, is added to the auxiliary substance(s). As a rule, the antigen is emulsified in this connection.

In the case of this embodiment, rabbits are particularly suitable for use as the host. The immunization cocktails are injected, preferably subcutaneously. Four injections, given at intervals of in each case five weeks, have proved to be advantageous. The antibody titers can be determined by means of an immunoassay, for example competitively, using a sheep antiserum which is directed against host IgG and labeled haptoglobin. In this way, it can be decided, toward the end of the immunization, whether a particular host is suitable for isolating antibody. If, for example, four immunizations are carried out, it is then possible to determine the antibody titer after the third immunization and then isolate antibodies from animals which exhibit an adequate antibody titer.

In order to obtain the antibodies which have been formed, preference is given to withdrawing blood from the hosts over a period of several weeks or months. In conclusion, the host can be exsanguinated. Serum, which contains the desired antibodies, can be isolated, in a known manner, from the blood which has been obtained. If necessary, the resulting complete serum can be purified, in a manner known to a skilled person, in order to concentrate the antibody fraction, and in particular the haptoglobin-specific antibodies, contained therein. It is also possible to obtain monoclonal haptoglobin-specific antibodies in this way. However, it is preferred, for this, to remove spleen tissue from the hosts and use the spleen lymphocytes which have been obtained in this way to establish, in customary manner, hybridomas which produce monoclonal antibodies.

The present invention also relates to the immunization cocktails which have been described in connection with the process according to the invention. These cocktails contain, where appropriate in addition to the other auxiliary substances, protein and at least one protein fragment and also, if necessary, adjuvant, preferably in the above-described formulations.

The present invention also relates to antisera which can be obtained by means of the above processes. These sera can be complete sera, i.e. blood which has been obtained from the host after the cellular and coaguable constituents have been separated off, or fractions of this serum in which, in particular, the immunoglobulin fraction, and preferably the haptoglobulin-specific immunoglobulin fraction, has been enriched. These fractions can be obtained using the methods which have been described above in connection with the antibody purification.

The antisera according to the invention are polyclonal; i.e. they contain antibodies of differing specificity, as a rule of differing classes and subclasses; normally, all the L chain isotypes are represented, and several protein epitopes are recognized.

Antisera according to the invention are crossreactive. In particular, they are specific for more than one haptoglobin and preferably specific for haptoglobin from at least two animal species, in particular productive animal and domestic animal species. Examples of particular embodiments are antisera which exhibit specificity for haptoglobin from pigs and horse or for haptoglobin from horse, dog and bovine. In this connection, specificity means the possibility of being able to detect a particular haptoglobin with an adequate degree of sensitivity. As was explained at the outset, the sensitivity which is required may differ from animal species to animal species, depending on the concentration at which haptoglobin occurs in the healthy animal or in the diseased animal. The antisera according to the invention advantageously exhibit sensitivities of less than 1 mg/ml of sample, preferably of less than 100 μg/ml of sample and particularly preferably of less than 10 μg/ml of sample. This means that the antiserum according to the invention can be used to detect at least the concentration of haptoglobin per ml of sample which is in each case specified, and advantageously lower concentrations as well.

The present invention also relates to the use of such a crossreactive antiserum for determining haptoglobin in productive animal and domestic animal samples. As a rule, the samples are body fluids, for example blood, plasma, serum, saliva, milk and the like. However, it is also possible to determine haptoglobin in other samples derived from productive animals and domestic animals. Meat juice may be mentioned as an the example in this connection.

The determination is effected immunologically. In principle, this can be carried out using any analytical or diagnostic test method in which antibodies are employed. The methods include agglutination and precipitation techniques, immunoassays, immunohistochemical methods and immunoblot techniques, e.g. Western blotting.

According to the invention, preference is given to use in immunoassays. Competitive immunoassays, i.e. antigen and labeled antigen (tracer) compete for binding the antibody, and Sandwich immunoassays, i.e. the binding of specific antibodies to the antigen is detected using a second, usually labeled antibody, are both suitable. These assays can be either homogeneous, i.e. without any separation into solid phase and liquid phase, or heterogeneous, i.e. bound labels are separated from unbound labels, for example using solid phase-bound antibodies. Depending on the label and the measurement method, the different heterogeneous and homogeneous immunoassay formats can be assigned to particular classes, for example RIAs (radioimmunoassays), ELISA (enzyme-linked immunosorbent assay), FIA (fluorescence immunoassay), LIA (luminescence immunoassay), TRFIA (time-resolved FIA), IMAC (immunoactivation), EMIT (enzyme-multiplied immune test) and TIA (turbodimetric immunoassay).

Competitive immunoassays are preferred for the haptoglobin determination according to the invention. In these assays, labeled haptoglobin (tracer) competes with the sample haptoglobin to be quantified for binding to the antiserum employed. A standard curve is then used to determine the quantity of antigen in the sample from the quantity of tracer which is displaced.

From the labels which are available for these purposes, enzymes have proved to be advantageous. For example, it is possible to use systems based on peroxidases, in particular horse radish peroxidase, alkaline phosphatase and β-D-galactosidase. Specific substrates, whose conversion can be monitored photometrically, for example, are available for these enzymes. Suitable substrate systems are based on p-nitrophenyl phosphate (p-NPP), 5-bromo-4-chloro-3-indolyl phosphate/Nitro Blue Tetrazolium (BCIP/NPT), or Fast Red/Naphthol AS-TS phosphate, in the case of alkaline phosphatase; 2,2-azinobis(3-ethylbenzthiazoline-6-sulfonic acid) (ABTS), o-phenylenediamine (OPT), 3,3′,5,5′-tetramethylbenzidine (TMB), o-dianisidine, 5-aminosalicylic acid, 3-dimethylaminobenzoic acid (DMAB) and 3-methyl-2-benzothiazoline hydrazone (MBTH), in the case of peroxidases; o-nitrophenyl-β-D-galactoside (o-NPG), p-nitrophenyl-β-D-galactoside and 4-methylumbelliphenyl-β-D-galactoside (MUG), in the case of β-D-galactosidase. In many cases, these substrate systems can be obtained commercially in ready-to-use form, for example in the form of tablets, which can also contain other reagents, such as expedient buffers and the like.

Labeled haptoglobin is used as a tracer. In conformity with this, it is possible, for the purpose of determining haptoglobin in a particular animal species, to label the haptoglobin which is to be determined and use it as the tracer. According to the invention, preference is given, for the purpose of preparing the tracer, to using an antigen which can be employed when determining haptoglobin derived from at least two productive animal and domestic animal species. In this respect, the antigen can be described as being animal species-nonspecific, and a tracer which is based on this antigen can be designated a multispecies tracer. The affinity of the binding of these multispecies tracers to the antiserum employed in the immunoassay is expediently selected such that haptoglobins derived from the animal species being investigated are able to displace the multispecies tracer. According to the invention, bovine haptoglobin, which is labeled with biotin, in particular, can be used as the multispecies tracer.

Labels can be coupled to haptoglobin, for the purpose of preparing tracers, in a manner known per se. The reader is referred, mutatis mutandis, to the above remarks with regard to coupling haptoglobin to protein fragments. In addition to this, a number of labels which are expediently modified for conjugation to proteins are available, for example biotin-conjugated, avidin-conjugated, extravidin-conjugated or streptavidin-conjugated enzymes, maleimide-activated enzymes, and the like. To form the tracer, these labels can be either reacted directly with haptoglobin or, if necessary, with haptoglobin which has been appropriately derivatized. If, for example, a streptavidin-peroxidase conjugate is used, this first of all requires the haptoglobin to be biotinylated. The same applies, in a corresponding manner, to the reverse arrangement. The skilled person is familiar with suitable methods for this purpose as well, for example, haptoglobin can be reacted with biotinamidocarboxylic acid N-hydroxysuccinimide ester, for example the caproate ester, biotin hydrazide, N-hydroxysuccinimidobiotin or 3-(N-maleimidopropionyl)biocitin, in order to be coupled to avidin-conjugated, streptavidin-conjugated, extravidin-conjugated or anti-biotin-conjugated labels.

According to a particular embodiment of the present invention, as tracer a bovine haptoglobin, which is biotinylated and which is coupled to a streptavidin-peroxidase conjugate, is used as the tracer.

If a heterogeneous immunoassay format is selected, the antigen-antibody complex can, for the purpose of separation, be bound to the support by way of an antiidiotypic antibody which is coupled to the support, for example an antibody which is directed against rabbit IgG. Supports, in particular microtiter plates, which are coated with appropriate antibodies are known and in some cases commercially available.

If necessary, hemoglobin is added to the test system. The quantity is as a rule dimensioned such that all the hemoglobin-binding sites on the haptoglobin which is present are saturated. This is advantageous when determining haptoglobin in strongly hemolytic samples and whole blood.

The determination of haptoglobin is used, in particular, for diagnosing infections, inflammations, traumas (tissue injuries) or immunological stress. It can be indicated in the case of acutely sick animals, be used for monitoring therapy and in stock management, be of assistance when optimizing housing conditions, and be used as an early indicator of the success of a vaccination, since the concentration of haptoglobin correlates with the antibody titer in the plasma of the vaccinated individual only a short time, as a rule 24 hours, after the vaccination.

The process according to the invention is also suitable for determining haptoglobin quantitatively in samples which have been stored over a long period (e.g. more than 2 years at −18° C.), in samples which had been stored inappropriately (e.g. 5 days at room temperature) and in heat-inactivated samples (e.g. 30 minutes at 56° C.).

The present invention also relates to immunoassay sets which contain at least one haptoglobin-specific antiserum, as described above, and additional components. These sets are compilations, as a rule pack units, of agents for implementing a haptoglobin determination according to invention. In order to make handling as simple as possible, these agents are preferably provided in what is essentially a ready-to-use form. An advantageous arrangement is the immunoassay in kit form. As a rule, a kit comprises several receptacles for arranging components separately. All the components can be provided in ready-to-use dilution, as concentrates for diluting, or as dry substances or lyophilizates for being dissolved or suspended, individual components, or all the components, can be frozen or stored at ambient temperature until used. Sera are preferably quick-frozen, for example at −20° C., which means that, in these cases, immunoassay preferably has to be kept at freezing temperature prior to use.

Other components which are included with the immunoassay depend on the nature of the immunoassay. As a rule, standard protein, any tracers which may be required, and control serum, are included together with the antiserum. Furthermore, it is possible to include microtiter plates, which are preferably coated with antibody, buffers, for example for the testing, for the washing or for the reaction of the substrate, and the enzyme substrate itself.

The following examples are intended to explain the above-described invention in more detail without restricting it.

### EXAMPLE 1

Haptoglobin Purification

8 ml of a saturated solution of (NH₄)₂SO₄ were added to 20 ml of a non-hemolytic serum (pig, bovine, horse or dog) thereby giving rise to a degree of saturation with (NH₄)₂SO₄—of about 30%. In order to prepare the saturated (NH₄)₂SO₄ solution, 41.5 g of (NH₄)₂SO₄ were dissolved in 50 ml of distilled water.

The ammonium sulfate-treated serum was stirred at room temperature for 1 hour. The sample was then centrifuged (3 200 g) at room temperature for 20 minutes. 12 ml of the saturated ammonium sulfate solution were added to the supernatant (approx. 25 ml). This gives a saturation of in all approx. 60%. The sample was once again stirred at room temperature for 1 hour. The subsequent centrifugation was carried out as described above. The resulting pellet was dissolved in 10 ml of Tris buffer (0.1 M Tris/HCl, 0.5 M NaCl, pH 7.0) and dialyzed overnight at 4° C. against Tris buffer.

The subsequent purification was effected by means of chromatography. Eluted fractions were analyzed photometrically, by determining the extinctions at 280 nm, 260 nm and 536 nm. The extinction coefficient of human haptoglobin (Sigma H7605) at 280 nm in PBS was used as the basis for determining the haptoglobin concentration. The extinction coefficient is 1.45 and was determined in PBS.

Bovine hemoglobin (Sigma H 2500) was first of all covalently coupled to activated CNBr-Sepharose (Amersham Pharmacia Biotech). The resulting material was loaded into a chromatography column (Amersham Pharmacia Biotech C10/10), which was equilibrated with the Tris buffer. The flow rate through the column was 1 ml/min. The dialysate (approx. 20 ml) from the above-described precipitation was loaded onto the column after having been filtered through a 0.45 μm-filter. Non-binding constituents were first washed out with about 40 ml of 0.1 M Tris buffer. This was followed by a further washing step with 12 ml of 1.6 M guanidine hydrochloride. The haptoglobin was then eluted with 3.5 M guanidine hydrochloride. The haptoglobin-containing fractions were obtained after a flow through of about 6 ml. The fraction size was 1 ml, with approx. 5 fractions being pooled. The sample was dialyzed overnight at 4° C. against PBS.

A gel filtration through Superdex 200® was carried out in a C16/70 column (Amersham Pharmacia Biotech) in order to separate off any remaining impurities.

The yield of haptoglobin depended mainly on the haptoglobin concentration in the sera which were used for the purification. For example, it was possible to obtain 4.8 mg of haptoglobin from pig serum, 0.6 mg from bovine serum, 1.5 mg from horse serum and 1.6 mg from dog serum.

### EXAMPLE 2

Immunization

(1) Composition of the Immunization Cocktails

Immunization cocktails were prepared which contained a mixture of haptoglobins from different animal species and two synthetic peptides. The following stock solutions were prepared:

Origin and Concentration of the Stock Solutions

| Haptoglobin | | Concentration of the stock |
|---|---|---|
| derived from: | Origin | solution |
| Pig (p) | Example 1 | 400 μg/ml |
| Bovine (b) | Example 1 | 150 μg/ml |
| | | 180 μg/ml |
| Horse (e) | Example 1 | 250 μg/ml |
| | | 250 μg/ml |
| Dog (d) | Example 1 | 200 μg/ml |
| Human (h) | Sigma | 1 mg/ml |
| Peptides | | |
| HPT-1 (SEQ ID NO: 1) | synthetic | 1 mg/ml |
| HPT-2 (SEQ ID NO: 2) | synthetic | 1 mg/ml |

The following haptoglobin mixtures were prepared using these stock solutions:

1. Initial Immunization

| Haptoglobin | Quantity | |
|---|---|---|
| derived from | μl | μg |
| Pig | 750 | 300 |
| Bovine | 1500 | 225 |
| Horse | 1200 | 300 |
| Dog | / | / |
| Human | 300 | 300 |
| Total | 3750 | 1125 |

2nd and 3rd Immunization

| Haptoglobin | Quantity | |
|---|---|---|
| derived from | μl | μg |
| Pig | 250 | 100 |
| Bovine | / | / |
| Horse | 400 | 100 |
| Dog | 500 | 100 |
| Human | 100 | 100 |
| Total | 1250 | 400 |

4th Immunization

| Haptoglobin | Quantity | |
|---|---|---|
| derived from | μl | μg |
| Pig | 190 | 75 |
| Bovine | 417 | 75 |
| Horse | 126 | 75 |
| Dog | 375 | 75 |
| Human | 75 | 75 |
| Total | 1180 | 375 |

The following reaction solutions (in μl) were subsequently prepared, incubated at room temperature for two hours separately from each other, then combined with each other and incubated at room temperature for a further hour (GA=glutaraldehyde).

1. Initial Immunization

| | HPT-1 | HPT-2 | pbehHPT | KLH | GA (2%) |
|---|---|---|---|---|---|
| 1) HPT-1-KLH | 200 | | | 400 | 50 |
| 2) HPT-2-KLH | | 200 | | 400 | 50 |
| 3) pbehHPT | | | 1250 | | |
| 4) HPT-1-HPT | 50 | | 1250 | | 12.5 |
| 5) HPT-2-HPT | | 50 | 1250 | | 12.5 |

2nd Immunization

| | HPT-1 | HPT-2 | pedhHPT | KLH | GA (2%) |
|---|---|---|---|---|---|
| 1) HPT-1-KLH | 100 | | | 200 | 25 |
| 2) HPT-2-KLH | | 100 | | 200 | 25 |
| 3) pedhHPT | | | 415 | | |
| 4) HPT-1-HPT | 25 | | 415 | | 12.5 |
| 5) HPT-2-HPT | | 25 | 415 | | 12.5 |

3rd Immunization

| | HPT-1 | HPT-2 | pedhHPT | KLH | GA (2%) |
|---|---|---|---|---|---|
| 1) pedhHPT | | | 415 | | |
| 2) HPT-1-HPT | 25 | | 415 | | 12.5 |
| 3) HPT-2-HPT | | 25 | 415 | | 12.5 |

4th Immunization

| | HPT-1 | HPT-2 | pbedhHPT | KLH | GA (2%) |
|---|---|---|---|---|---|
| 1) pbedhHPT | | | 400 | | |
| 2) HPT-1-HPT | 25 | | 400 | | 12.5 |
| 3) HPT-2-HPT | | 25 | 400 | | 12.5 |

The resulting solutions were then emulsified in complete Freund's adjuvant (7 ml, 4 ml, 3 ml and 3 ml, respectively), aliquoted out (4×3 ml, 4×1.5 ml, 4×1.5 ml and 3×1.5 ml, respectively) and injected subcutaneously into rabbits.

In all, four rabbits were subjected to four immunizations at 5-weekly intervals. The components which were administered together with immunization cocktails 1-4 are compiled below:

Quantities of the Individual Constituents in Immunization Cocktails 1 to 4:

| Constituent | | | | |
|---|---|---|---|---|
| Haptoglobin | Quantity per rabbit (μg) | | | |
| derived from | Initial | 2nd | 3rd | 4th |
| Pig | 75 | 25 | 25 | 25 |
| Bovine | 56 | / | / | 25 |
| Horse | 75 | 25 | 25 | 25 |
| Dog | / | 25 | 25 | 25 |
| Human | 75 | 25 | 25 | 25 |
| Peptide | | | | |
| HPT-1 | 62.5 | 31.25 | 6.25* | 6.25* |
| HPT-2 | 62.5 | 31.25 | 6.25* | 6.25* |

*only coupled to haptoglobin

An antibody titer determination was performed after the 3rd immunization.

A competitive ELISA was used for determining this antibody titer. Microtiter plates were coated with coating buffer (Na carbonate, pH 9.6) containing 5 μg of ovine IgG/ml and incubated at 4° C. overnight. The antibodies were obtained from sheep which had been immunized against rabbit IgG. The antibodies contained in the sheep serum were isolated by affinity chromatography. The plates were subsequently saturated with 2.5% casein solution at room temperature for two hours. After the plates had been washed five times with washing buffer, it was then possible to apply the antiserum to be tested. The antiserum was applied in various dilution steps of from 1:10 000 to 1:100 000 in hemoglobin-containing test buffer (1 mg of hemoglobin/ml). Biotin-labeled haptoglobin was then pipetted onto the plates at a dilution of 8 ng/ml. After the plates had been incubated at room temperature for one hour, they were then washed three times. In the next step, the plates were incubated for 30 minutes with streptavidin-peroxidase. The plates were subsequently washed five times. TMB solution was used as a substrate, the color reaction was stopped with 2 M H₂SO₄ after 20 minutes. The extinction as determined at 450 nm in a plate photometer. The antibody titer was defined as being the antiserum dilution which gave a net extinction of 1 in the test system employed.

It was consequently possible to use this test to specifically detect all the rabbit IgGs which were directed against haptoglobin.

Because its titer development was low, one rabbit was removed from the experiment. It was possible to obtain high-titer antisera from the remaining rabbits. The weekly removal of blood began one week after the 3rd immunization.

### EXAMPLE 3

Using the Antisera in the Immunoassay

An investigation was carried out into the ability of the antisera obtained in example 2 to bind to haptoglobin derived from pig, bovine, horse, dog and human. The antisera were observed to crossreact with all the haptoglobins tested. In addition to this, it was also possible to determine haptoglobin in cat serum, even though the immunization cocktail which was used did not contain any purified cat haptoglobin.

It was possible to detect pig haptoglobin sensitively in a concentration range of from 30 to 1 000 ng/ml of serum.

In addition to this, it was found that the concentrations in whole blood correlated statistically significantly with the serum concentrations (r=0.85, p<0.001) and, at the same time, were about 40% lower than those in the serum.

In principle, it is also possible to determine the haptoglobin concentrations in saliva and meat juice samples. The concentrations in these samples are markedly lower than in serum, being about 1 000-fold lower in saliva and about 10-fold lower in meat juice.

1. A process for preparing protein-specific antibodies by immunizing a host and, in a manner known per se, obtaining the antibodies, with at least one immunization cocktail, which contains protein and at least one fragment thereof, being administered to the host, characterized in that protein from at least two different animal species is used.

2. The process as claimed in claim 1, characterized in that at least a part of the protein fragment(s) is coupled to at least a part of the protein.

3. The process as claimed in claim 1 or 2, characterized in that the protein fragment is a peptide having a length of from 10 to 30 amino acids.

4. The process as claimed in one of the preceding claims, characterized in that at least one protein fragment which exhibits conserved sequences is used.

5. A process for preparing haptoglobin-specific antibodies by immunizing a host and in known per se obtaining the antibodies, characterized in that at least one immunization cocktail, which contains haptoglobin and at least one haptoglobin fragment, is administered to the host.

6. The process as claimed in claim 5, characterized in that haptoglobin from pig, bovine, horse, dog and/or human is used.

7. The process as claimed in claim 5 or 6, characterized in that haptoglobin peptides having the amino acid sequences SEQ ID NO:1 and/or SEQ ID NO:2 are used.

8. An immunization cocktail which is composed of protein, at least one fragment thereof and, where appropriate, auxiliary substances, characterized in that protein from at least two different animal species is used.

9. A peptide having one of the amino acid sequences SEQ ID NO:1 and SEQ ID NO:2.

10. A polyclonal, crossreacted antiserum which has specificity for haptoglobin derived from at least two productive animal and domestic animal species and which can be obtained using a process as claimed in one of claims 5 to 8.

11. The use of a crossreactive antiserum as claimed in claim 10 for determining haptoglobin in productive animal and domestic animal samples.

12. The use as claimed in claim 11 for diagnosing infections, inflammations, traumas and immunological stress.

13. An immunoassay set which comprises at least one crossreactive antiserum as claimed in claim 10 and also additional components.
